(19) United States
(12) Patent Application Publication
(10) Pub. No.: US 2012/0083713 A1
(43) Pub. Date: Apr. 5, 2012

(54) DEVICE FOR GAUGING A FORCE THRESHOLD AND ENGAGING A TARGET OF INTEREST

(75) Inventors: Francis X. Creighton, JR., Atlanta, GA (US); Norman Wendell Todd, Atlanta, GA (US)

(73) Assignee: EMORY UNIVERSITY, Atlanta, GA (US)

(21) Appl. No.: 13/250,023

(22) Filed: Sep. 30, 2011

Related U.S. Application Data
(60) Provisional application No. 61/388,469, filed on Sep. 30, 2010.

(51) Int. Cl. A61B 5/103

(52) U.S. Cl. 600/587

(57) ABSTRACT

The disclosure generally relates to devices and instruments for engaging a target of interest, having a resilient member for gauging a force threshold. The device or instrument may include a tip portion, wherein the tip portion includes a first end portion a having an engaging member configured to engage a target of interest, an opposite, second end portion, and a body portion defined therebetween, the body portion including a resilient member configured to be elastically compressible up to a predetermined maximum compression level.

### CROSS REFERENCE TO RELATED APPLICATION

This application claims priority to U.S. Provisional Application Ser. No. 61/388,469, filed on Sep. 30, 2010 which application is hereby incorporated by this reference in its entirety.

### FIELD

The disclosure generally relates to devices and instruments for engaging a target of interest, having a resilient member for gauging a force threshold.

### BACKGROUND

Existing force gauges are complicated mechanical devices or electronic devices that may indicate a maximum force that is exerted upon a target of interest, but they do not allow for a user of an implement for applying a force to a target to readily observe and ensure that an applied amount of force does not exceed a predetermined maximum threshold. Also, such devices are not feasible to be used in applications requiring a small and technically precise implement, such as during certain types of surgery or placement of microprocessing components.

Existing force gauges are not feasible to be used in connection with, for example, surgical procedures involving a living subject, due their large size, cost, and inability to be sterilized. In a surgical procedure for installing a cochlear implant electrode array in a living subject, existing force gauges do not allow for a medical professional to readily observe and ensure that a predetermined maximum amount of force is not being exerted onto the electrode array while it is being inserted into the scala tympani. An amount of applied force that exceeds a predetermined safe level can cause damage to the cochlea of the living subject. There remains a need for a simple, readily-observable way to ensure that a force greater than a predetermined value is being exerted when performing precise procedures such as surgical implants, thus reducing the likelihood that damage will occur during implantation.

### SUMMARY

Therefore, a heretofore unaddressed need still exists in the art to address the aforementioned deficiencies and inadequacies.

According to embodiments, the disclosure relates to a device for engaging a target of interest. Typically, the device is configured to respond to a force measurement, and in particular embodiments to a force being exerted on an aspect of the device. In one embodiment, the device may include a base portion having a first end, an opposite second end portion, and a body portion defined therebetween. The device may also include a handle portion extending away from the second end portion of the base portion and substantially coaxial with the base portion, where the handle portion includes a first end portion, a second end portion, and a body portion defined therebetween. Further, the device may include a tip portion extending away from the first end portion of the base portion and substantially coaxial with the base portion, where the tip portion includes a first end portion having an engaging member configured to engage a target of interest, an opposite, second end portion, and a body portion defined therebetween. The body portion may include a resilient member configured to be elastically compressible up to a predetermined maximum compression level such that a force applied to at least one of the tip portion, base portion, and handle portion is up to or below a first predetermined maximum force level.

In some embodiments, the predetermined maximum compression level may correspond to a predetermined buckling limit of an elastic rod formed from a predetermined material and having a predetermined length and width, and where the resilient member may be of at least partially of the predetermined material and has a length and width that are substantially similar to the predetermined length and width of the elastic rod.

In some embodiments, the force applied to at least one of the tip portion, base portion, and handle portion is the net force in a direction substantially parallel to the longitudinal axis of the base portion, that is produced from a force applied to the handle portion in a first direction and a force applied to the tip portion in a second direction that substantially opposite the first direction.

In some embodiments, the resilient member may be configured such that at least one of the length and width is adjustable to change the predetermined maximum compression level.

In some embodiments, the tip portion may be configured such that the resilient member is selectively detachable from the tip portion and such that a different resilient member having a different predetermined maximum compression level is attachable in place of a detached resilient member.

In some embodiments, the base portion may include a first receptacle disposed inside along the longitudinal axis, the first receptacle having sidewalls defining a first inner cavity and a first opening at the end of the first end portion of the base portion. The first receptacle may be configured to receive at least part of the tip portion in the first inner cavity through the first opening. The base portion may further include a second receptacle disposed inside along the longitudinal axis, the second receptacle having sidewalls defining a second inner cavity and a second opening at the end of the second end portion of the base portion. The second receptacle may be configured to receive at least part of the handle portion in the second inner cavity through the second opening.

In another embodiment, the target of interest may include at least a portion of a surgical prosthesis to be placed in a target area of a living subject. The target area may include a predetermined region of the inner ear of a living subject and the portion of the surgical prosthesis includes an electrode array for a cochlear implant.

In some embodiments, the target of interest may include at least a portion of a surgical prosthesis that has been placed in a target area of a living subject. The target area may include a predetermined region of the inner ear of a living subject and the portion of the surgical prosthesis includes an electrode array for a cochlear implant.

According to some embodiments, the disclosure may relate to a surgical instrument. In some embodiments, the surgical instrument may include a base portion having a first end portion, an opposite second end portion, and a body portion defined therebetween. The surgical instrument may also include a handle portion extending away from the second end portion of the base portion and substantially coaxial with the base portion. The handle portion may include a first end, a second end, and a body portion defined therebetween. Further, the surgical instrument may include a tip portion extending away from the first end portion of the base portion in a first direction at a predetermined angle relative to the longitudinal axis of the base portion. The tip portion may include a first end portion having an engaging member configured to engage a target of interest, an opposite, second end portion, and a body portion defined therebetween. The body portion may include a resilient member configured to be elastically compressible up to a predetermined maximum compression level such that a force applied to at least one of the tip portion, base portion, and handle portion is up to or below a first predetermined maximum force level. The base portion may include a first receptacle disposed inside along the longitudinal axis, the first receptacle having sidewalls defining a first inner cavity and a first opening at the end of the first end portion of the base portion, where the first receptacle is configured to receive at least part of the tip portion inside the first inner cavity through the first opening. The base portion may further include a second receptacle disposed inside the base portion along the longitudinal axis, the second receptacle having sidewalls defining a second inner cavity and a second opening at the end of the second end portion of the base portion, the second receptacle configured to receive at least part of the handle portion inside the second inner cavity through the second opening.

In some embodiments, the predetermined maximum compression level may correspond to a predetermined buckling limit of an elastic rod formed from a predetermined material and having a predetermined length and width. The resilient member may be formed at least partially of the predetermined material and is formed with a length and width that are substantially similar to the predetermined length and width of the elastic rod.

In some embodiments, the resilient member may be configured such that at least one of the length and width is adjustable to change the predetermined maximum compression level.

In some embodiments, the force applied to the tip portion, base portion, and handle portion may be the net force in a direction substantially parallel to the longitudinal axis of at least one of the base portion, tip portion, and handle portion that is produced from a force applied to the handle portion in a first direction and a force applied to the tip portion in a second direction that is substantially opposite the first direction.

In some embodiments, the predetermined angle may be in a range from about 0 to about 90 degrees. In other embodiments, the predetermined angle may be in a range from about 90 degrees to about 180 degrees.

In some embodiments, the tip portion may be configured such that the resilient member is selectively detachable from the tip portion and such that a different resilient member having a different predetermined maximum compression level is attachable in place of a detached resilient member.

In some embodiments, the target of interest may include at least a portion of a surgical prosthesis to be placed in a target area of a living subject and the target area includes a predetermined region of the inner ear of a living subject. The portion of the surgical prosthesis includes an electrode array for a cochlear implant.

In some embodiments, the target of interest may include at least a portion of a surgical prosthesis that has been placed in a target area of a living subject, and the target area includes a predetermined region of the inner ear of a living subject. The portion of the surgical prosthesis may include an electrode array for a cochlear implant.

In other embodiments, the present disclosure relates to a surgical instrument for engaging a target of interest that is placeable in a target area of a living subject. In some embodiments, the surgical instrument may include a base portion having a first end portion, an opposite second end portion, and a body portion defined therebetween. The surgical instrument may also include a handle portion extending away from the second end portion of the base portion and substantially coaxial with the base portion, where the handle portion includes a first end portion, a second end portion, and a body portion defined therebetween. Further, the surgical instrument may include a tip portion extending away from the first end portion of the base portion in a first direction at a predetermined angle relative to the longitudinal axis of the base portion. The tip portion may include a first end portion having an engaging member configured to engage a target of interest in a target area. The target of interest may include a surgical prosthesis and the target area includes a predetermined region of interest within a living subject. The tip portion may also include a second end portion and a body portion defined between the first end portion and the second end portion. The body portion may include a resilient member configured to be elastically compressible up to a predetermined maximum compression level such that a force applied to the tip portion, base portion, and handle portion is up to or below a first predetermined maximum force level.

In some embodiments, the target area may include a predetermined region of the inner ear of a living subject and the surgical prosthesis includes a cochlear implant.

In some embodiments, the force applied to the tip portion, base portion, and handle portion may be the net force in a direction substantially parallel to the longitudinal axis of at least one of the base portion, tip portion, and handle portion that is produced from a force applied to the tip portion in a first direction and a force applied to the handle portion in a second direction that is substantially opposite the first direction.

In some embodiments, the predetermined maximum compression level may correspond to a predetermined buckling limit of an elastic rod formed from a predetermined material and having a predetermined length and width, and where the resilient member is formed at least partially of the predetermined material and has a length and width that are substantially similar to the predetermined length and width of the elastic rod.

In some embodiments, the resilient member may be configured such that at least one of the length and width is adjustable to change the predetermined maximum compression level.

In some embodiments, the tip portion may be configured such that the resilient member is selectively detachable from the tip portion and such that a different resilient member having a different predetermined maximum compression level is attachable in place of a detached resilient member.

In some embodiments, the predetermined angle may be about 90 degrees.

In some embodiments, the body portion may include a first receptacle disposed inside along the longitudinal axis, the first receptacle having sidewalls defining a first inner cavity and a first opening at the end of the first end portion of the body portion. The first receptacle may be configured to receive at least part of the tip portion in the first inner cavity through the first opening. The base portion may further include a second receptacle disposed inside along the longitudinal axis, the second receptacle having sidewalls defining a second inner cavity and a second opening at the end of the second end portion of the base portion, the second receptacle configured to receive at least part of the handle portion in the second inner cavity through the second opening.

In some embodiments, at least the second end portion of the tip portion may be dimensioned to be complimentary to the dimensions of the first receptacle. In some embodiments, at least the first end portion of the handle portion may be dimensioned to be complimentary to the dimensions of the second receptacle.

In some embodiments, the target area may include a predetermined region of the inner ear of a living subject and the surgical prosthesis may include a cochlear implant.

In some embodiments, at least the body portion of the handle portion may be configured to be grasped by a user.

These and other aspects of the present disclosure will become apparent from the following description of the preferred embodiments, taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments and, together with the written description, serve to explain the principles of the disclosure. Wherever possible, the same reference numbers are used throughout the drawings to refer to the same or like elements of an embodiment, and wherein:

FIGS. 1A-C show schematically a partial view of a device for engaging a target of interest, wherein FIG. 1A shows a base portion of the device, FIG. 1B shows a tip portion of the device, and FIG. 1C shows a handle portion of the device, according to one embodiment of the present disclosure;

FIG. 2 shows schematically partial views of a base portion and a tip portion in a first configuration, according to one embodiment of the present disclosure;

FIG. 3 shows schematically partial views of a base portion and a tip portion in a second configuration, according to one embodiment of the present disclosure;

FIG. 4 shows schematically a partial view of a tip portion according to one embodiment of the present disclosure;

FIG. 5 shows schematically partial views of a device for engaging a target of interest according to one embodiment of the present disclosure;

FIG. 6 shows schematically a partial view of a handle portion according to one embodiment of the present disclosure;

FIG. 7 shows a plot of a correlation of first quantified values relating to a resilient member according to one embodiment of the present disclosure; and

FIG. 8 shows a plot of a correlation of second quantified values relating to a resilient member according to one embodiment of the present disclosure.

### DETAILED DESCRIPTION DISCLOSURE

### Definitions

The present disclosure is more particularly described in the following examples that are intended as illustrative only since numerous modifications and variations therein will be apparent to those skilled in the art. Various embodiments are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise.

It will be understood that the devices and instruments according to embodiments do require all portions described herein. In some embodiments, the devices and instruments may include any number of portions described herein. For example, the devices and instruments may only include the tip portion.

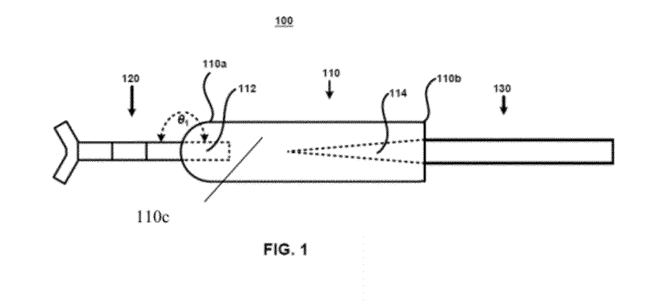

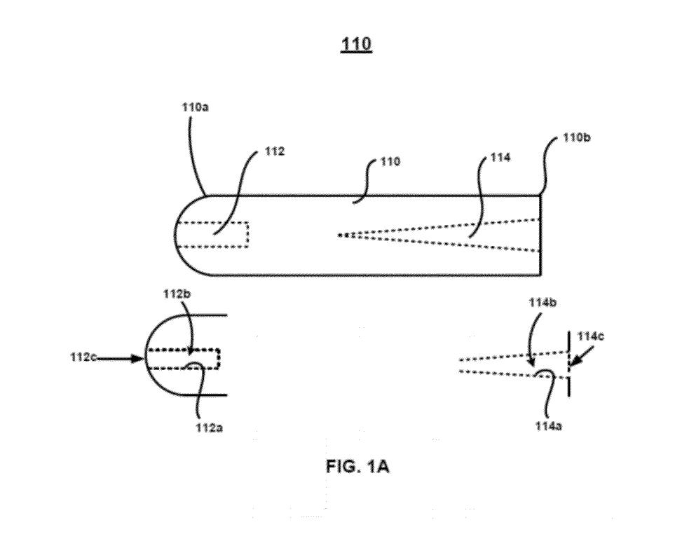

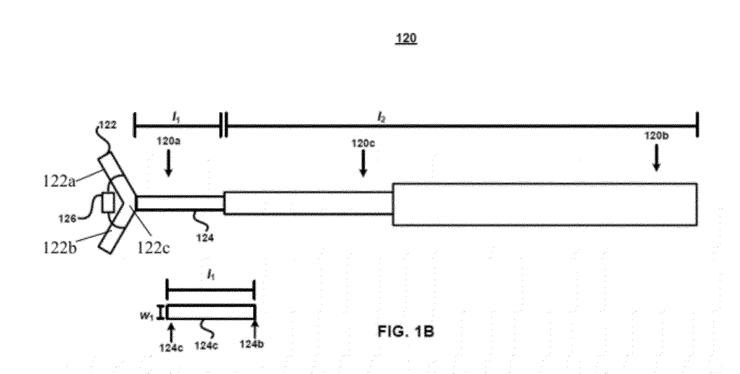

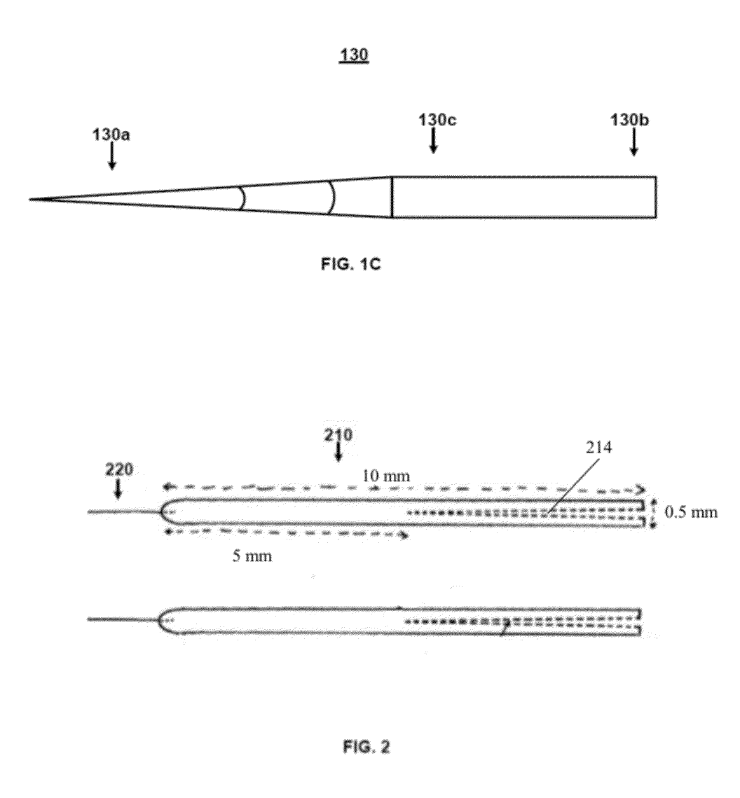

The description will be made as to the embodiments of the present disclosure in conjunction with the accompanying drawings in FIGS. 1-6. Now referring to FIGS. 1A, 1B, and 1C, in some embodiments, the disclosure relates to a device 100 for engaging a target of interest 126.

### Device

In one embodiment, the device 100 may include a base portion 110 having a first end portion 110a, an opposite second end portion 110b, and a body portion 110c defined therebetween.

The device 100 may also include a handle portion 130 extending away from the second end portion 110b of the base portion 110 and substantially coaxial with the base portion 110, where the handle portion 130 includes a first end portion 130a, a second end portion 130b, and a body portion 130c defined therebetween.

Further, the device 100 include a tip portion 120 extending away from the first end portion 110a of the base portion 110 and substantially coaxial with the base portion 110. The tip portion 120 may include a first end portion 120a, an opposite, second end portion 120b, and a body portion 120c defined therebetween. The tip portion 120 may include an engaging member 122 that is configured to engage a target of interest 126. The first end portion 120a may include the engaging member 122. The engaging member 122 may include a first portion 122a and a second portion 122b. The first and second portions 122a and 122b may converge at a third portion 122c. The third portion 122c may be disposed adjacent to the first end portion 120a.

The engaging member 122 may be made of an elastically deformable material. The material may include but is not limited to biocompatible plastics, polymers, as well as deformable metal, such as copper, aluminum, iron, etc.

The tip portion 120 may further include a resilient member 124 configured to be elastically compressible up to a predetermined maximum compression level such that a force applied to at least one of the tip portion 120, base portion 110, and handle portion 130 may be up to or below a first predetermined maximum force level. In some embodiments, the body portion 120c may include all or part of the resilient member 124. In other embodiments, the first end portion 120a may include all or part of the resilient member 124.

The resilient member 124 may be substantially coaxial with the main body portion 120c. The resilient member 124 may include a first end portion 124a, an opposite, second end portion 124b, and a body portion 1204c defined therebetween.

In some embodiments, the resilient member 124 may be an elastic rod. The resilient member 124 may be formed of the same or different material as the engaging member 122. The engaging member 122 may be made of an elastically deformable material. The material may include but is not limited to biocompatible plastics, polymers, as well as deformable metal, such as copper, aluminum, iron, etc. In some embodiments, the resilient member 124 may be formed of any polymer that is highly elastic.

Typically, the resilient member is made of a material with a Young's Modulus that, when input in the following formula (Euler's formula), will result in the desired “maximum” or “buckling” force.

$$F max = \frac{{π}^{2} \times E \times I}{{( k \times 1 )}^{2}}$$

$$I = \frac{π \times {d}^{4}}{64}$$

$$k = 0.5 \\ ∴ F max = \frac{{π}^{2} \times E \times {d}^{4}}{16 \times {l}^{2}}$$

when l=length of rod; E=Young's Modulus; Fmax=Buckling Force (i.e. the Maximum Force that will cause the rod to buckle); d=diameter of rod, I=area moment of inertia (for a cylindrical rod, I=(π3×d4)/64) and k=effective length constant. For a rod with one fixed and one free end, k is typically equal to 0.5.

Young's modulus can typically be calculated by dividing the tensile stress by the tensile strain in the elastic portion (linear) of a stress-strain curve of a material. In some embodiments, the resilient member 124 may be formed of a material having a Young's modulus about 1-5 GPa. In other embodiments, the material has a Young's modulus of less than about 2 GPa, or less than about 3 GPa, or less than about 4 GPa, or less than about 5 GPa. In certain embodiments, the material is a polymer such as PVC, piolystyrene, PTFE, Polypropylene, Nylon, Cellulose Nitrate, Cellulose Acetate, Acrylic, or a Polyethylene

In another embodiment, the predetermined maximum compression level may correspond to a predetermined buckling limit of an elastic rod formed from a predetermined material and having a predetermined length and width, and where the resilient member 124 is formed at least partially of the predetermined material. The resilient member 124 may have a length l₁ and width w₁ that are substantially similar to the predetermined length and width of the elastic rod. The length l₁ may be defined by the length of the body portion 124c. In one embodiment, the force applied to the tip portion 120, base portion 110, and/or handle portion 130 may be the net force in a direction substantially parallel to the longitudinal axis of the base portion 110, that is produced from a force applied to the handle portion 130 in a first direction and a force applied to the tip portion 120 in a second direction that is substantially opposite the first direction.

In one embodiment, the resilient member 124 may be configured such that at least one of the length l₁ and width w₁ is adjustable to change the predetermined maximum compression level.

In one embodiment, the tip portion 120 may be configured such that the resilient member 124 may be selectively detachable from the tip portion 120 and such that a different resilient member having a different predetermined maximum compression level may be attachable in place of a detached resilient member.

In one embodiment, the base portion 110 may include (or define) a first receptacle 112 disposed inside along the longitudinal axis. The first receptacle 112 may have sidewalls 112a defining a first inner cavity 112b and a first opening 112c at the end of the first end portion 110a of the base portion 110. The first receptacle 112 may be configured to receive at least part of the tip portion 120 in the first inner cavity 112b through the first opening 112c. The base portion 110 may further include (or define) a second receptacle 114 disposed inside along the longitudinal axis. The second receptacle 114 may have sidewalls 114a defining a second inner cavity 114b and a second opening 114c at the end of the second end portion 110b of the base portion 110. The second receptacle 114 may be configured to receive at least part of the handle portion 130 in the second inner cavity 114b through the second opening 114c.

In one embodiment, the target of interest 126 may include a target area of living subject. The target of interest 126 may further include at least a portion of a surgical prosthesis to be placed in a target area of a living subject. The target area may include but is not limited to a predetermined region of the inner ear of a living subject and the portion of the surgical prosthesis includes an electrode array for a cochlear implant. The target area may be any region for which a surgical prosthesis, such as the eye and an ocular implant.

In one embodiment, the target of interest 126 may include at least a portion of a surgical prosthesis that has been placed in a target area of a living subject. For example, the target area may include a predetermined region of the inner ear of a living subject and the portion of the surgical prosthesis includes an electrode array for a cochlear implant.

According to another aspect, the present disclosure may relate to a device or surgical instrument 100. In one embodiment, the surgical instrument may include a base portion 110 having a first end 110a, an opposite second end 110b, and a body portion 110c defined therebetween.

The surgical instrument 100 may also include a handle portion 130 extending away from the second end portion 110b of the base portion 110 and substantially coaxial with the base portion 110. The handle portion 130 may include a first end portion 130a, a second end portion 130b, and a body portion 130c defined therebetween.

The surgical instrument 100 may also include a tip portion 120 extending away from the first end portion 110a of the base portion 110 in a first direction at a predetermined angle θ₁ relative to the longitudinal axis of the base portion 110.

The tip portion 120 may include an engaging member 122 that is configured to engage a target of interest 126. The first end portion 120a may include the engaging member 122. The engaging member 122 may include a first portion 122a and a second portion 122b. The first and second portions 122a and 122b may converge at a third portion 122c. The third portion 122c may be disposed adjacent to the first end portion 120a.

The engaging member 122 may be made of an elastically deformable material. The material may include but is not limited to biocompatible plastics, polymers, as well as deformable metal, such as copper, aluminum, iron, etc.

The tip portion 120 may further include a resilient member 124. The resilient member 124 may be configured to be elastically compressible up to a predetermined maximum compression level such that a force applied to the tip portion 120, base portion 110, and/or handle portion 130 is up to or below a first predetermined maximum force level.

In some embodiments, the body portion 120c may include all or part of the resilient member 124. In other embodiments, the first end portion 120a may include all or part of the resilient member 124. The resilient member 124 may be substantially coaxial with the main body portion 120c. The resilient member 124 may include a first end portion 124a, an opposite, second end portion 124b, and a body portion 124c defined therebetween.

The base portion 110 may include or defines a first receptacle 112 disposed inside along the longitudinal axis, the first receptacle 112 having sidewalls 112a defining a first inner cavity 112b and a first opening 112c at the end of the first portion 110a of the base portion 110. The first receptacle 112 may be configured to receive at least part of the tip portion 120 inside the first inner cavity 112b through the first opening 112c. The base portion 110 may also include or define a second receptacle 114 formed inside along the longitudinal axis. The second receptacle 114 may have sidewalls 114a defining a second inner cavity 114b and a second opening 114c at the end of the second end portion 110b of the base portion 110. The second receptacle 114 may be configured to receive at least part of the handle portion 130 inside the second inner cavity 114b through the second opening 114c.

In one embodiment, the predetermined maximum compression level may correspond to a predetermined buckling limit of an elastic rod formed from a predetermined material and having a predetermined length and width. The resilient member 124 may be formed at least partially of the predetermined material and may have a length l₁ and width w₁ that are substantially similar to the predetermined length and width of the elastic rod.

In one embodiment, the resilient member 124 may be configured such that at least one of the length l₁ and width w₁ is adjustable to change the predetermined maximum compression level.

In one embodiment, the force applied to the tip portion 120, base portion 110, and/or handle portion 130 may be the net force in a direction substantially parallel to the longitudinal axis of at least one of the base portion 110, tip portion 120, and handle portion 130 that is produced from a force applied to the handle portion 130 in a first direction and a force applied to the tip portion 120 in a second direction that is substantially opposite the first direction.

In one embodiment, the predetermined angle θ₁ may be in a range from about 0 to about 90 degrees. In another embodiment, the predetermined angle θ₁ may be in a range from about 90 degrees to about 180 degrees.

In one embodiment, the tip portion 120 may be configured such that the resilient member 124 is selectively detachable from the tip portion 120 and such that a different resilient member having a different predetermined maximum compression level may be attachable in place of a detached resilient member.

In one embodiment, the target of interest 126 may include a target area of living subject. The target of interest 126 may further include at least a portion of a surgical prosthesis to be placed in a target area of a living subject. The target area may include but is not limited to a predetermined region of the inner ear of a living subject and the portion of the surgical prosthesis includes an electrode array for a cochlear implant. The target area may be any region for which a surgical prosthesis, such as the eye and an ocular implant.

In one embodiment, the target of interest 126 may include at least a portion of a surgical prosthesis that has been placed in a target area of a living subject, and the target area includes a predetermined region of the inner ear of a living subject. For example, the portion of the surgical prosthesis includes an electrode array for a cochlear implant.

According to yet another aspect, the present disclosure may relate to a surgical instrument or device 100 for engaging a target of interest 126 that is placeable in a target area of a living subject. In one embodiment, the surgical instrument may include a base portion 110 having a first end portion 110a, an opposite second end portion 110b, and a body portion 110c defined therebetween. The surgical instrument 100 may also include a handle portion 130 extending away from the second end portion 110b of the base portion 110 and substantially coaxial with the base portion 110, where the handle portion 130 includes a first end portion 130a, a second end portion 130b, and a body portion 130c defined therebetween.

The surgical instrument 100 may also include a tip portion 120 extending away from the first end portion 110a of the base portion 110 in a first direction at a predetermined angle θ₁ relative to the longitudinal axis of the base portion 110.

The tip portion 120 may include an engaging member 122 that is configured to engage a target of interest 126. The first end portion 120a may include the engaging member 122. The engaging member 122 may include a first portion 122a and a second portion 122b. The first and second portions 122a and 122b may converge at a third portion 122c. The third portion 122c may be disposed adjacent to the first end portion 120a.

The engaging member 122 may be made of an elastically deformable material. The material may include but is not limited to biocompatible plastics, polymers, as well as deformable metal, such as copper, aluminum, iron, etc.

The target of interest 126 may include a surgical prosthesis and the target area includes a predetermined region of interest within a living subject.

The tip portion 120 may also include a second end portion 120b and a body portion 120c defined between the first end portion 120a and the second end portion 120b. The tip portion 120 may include a resilient member 124 configured to be elastically compressible up to a predetermined maximum compression level such that a force applied to at least one of the tip portion 120, base portion 110, and handle portion 130 is up to or below a first predetermined maximum force level. In some embodiments, the body portion 120c may include all or part of the resilient member 124. In other embodiments, the first end portion 120a may include all or part of the resilient member 124.

In one embodiment, the target area may include a portion of a surgical prosthesis to be placed in a target area of a living subject and a portion of the region of the living subject where the surgical prosthesis is to be implanted. In some embodiments, the target area may include a predetermined region of the inner ear of a living subject and the surgical prosthesis includes a cochlear implant.

In one embodiment, the force applied to at least one of the tip portion 120, base portion 110, and handle portion 130 may be the net force in a direction substantially parallel to the longitudinal axis of at least one of the base portion 110, tip portion 120, and handle portion 130 that is produced from a force applied to the tip portion 120 in a first direction and a force applied to the handle portion 130 in a second direction that is substantially opposite the first direction.

In one embodiment, the predetermined maximum compression level may correspond to a predetermined buckling limit of an elastic rod formed from a predetermined material and having a predetermined length and width, and where the resilient member 124 may be formed at least partially of the predetermined material and may have a length l₁ and width w₁ that are substantially similar to the predetermined length and width of the elastic rod.

In one embodiment, the resilient member 124 may be configured such that at least one of the length hand width w₁ is adjustable to change the predetermined maximum compression level.

In one embodiment, the tip portion 120 may be configured such that the resilient member 124 is selectively detachable from the tip portion 120 and such that a different resilient member having a different predetermined maximum compression level may be attachable in place of a detached resilient member.

In one embodiment, the predetermined angle θ₁ may be about 90 degrees.

In one embodiment, the body portion 110 may include or define a first receptacle 112 disposed inside along the longitudinal axis. The first receptacle 112 may have sidewalls 112a defining a first inner cavity 112b and a first opening 112c at the end of the first end portion 110a of the body portion 110. The first receptacle 112 may be configured to receive at least part of the tip portion 120 in the first inner cavity 112b through the first opening 112c. The base portion 110 may also include a second receptacle 114 disposed inside along the longitudinal axis. The second receptacle 112 may have sidewalls 114a defining a second inner cavity 114b and a second opening 114c at the end of the second end portion 110b of the base portion. The second receptacle 114 being configured to receive at least part of the handle portion 130 in the second inner cavity 114b through the second opening 114c.

In one embodiment, at least the second end portion 120b of the tip portion 120 may be dimensioned to be complimentary to the dimensions of the first receptacle 112.

In one embodiment, at least the first end portion 130a of the handle portion 130 may be dimensioned to be complimentary to the dimensions of the second receptacle 114.

In one embodiment, the target area may include a portion of a surgical prosthesis to be placed in a target area of a living subject and a portion of the region of the living subject where the surgical prosthesis is to be implanted. In one embodiment, the target area may include a predetermined region of the inner ear of a living subject and the surgical prosthesis may include a cochlear implant.

In one embodiment, at least the body portion 130c of the handle portion 130 may be configured to be grasped by a user.

FIGS. 2-6 show examples of different embodiments of the device and instruments. FIG. 2 shows partial views of a base portion 210 and a tip portion 220 in a first configuration, according to one embodiment of the present disclosure. In this example, the width of the base portion may be about 0.5 mm. The total length of the base portion 210 may be about 10 mm and the total length of the tip portion 220 may be about 1.5 mm. The base portion 210 may have a length of 5 mm to a second receptacle 214. The view is the same from each side.

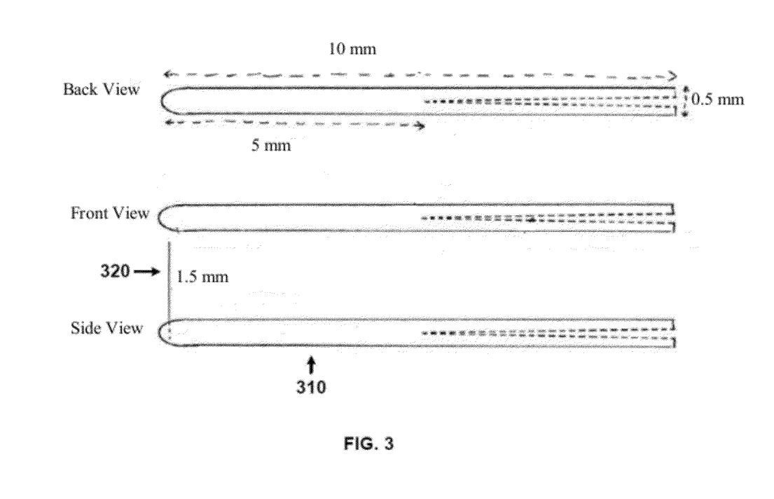

FIG. 3 shows partial views of a base portion 310 and a tip portion 320 in a second configuration, according to one embodiment of the present disclosure. In this example, the width of the base portion may be about 0.5 mm. The total length of the base portion 210 may be about 10 mm and the total length of the tip portion 220 may be about 1.5 mm. The base portion 210 may have a length of 5 mm to a second receptacle 214.

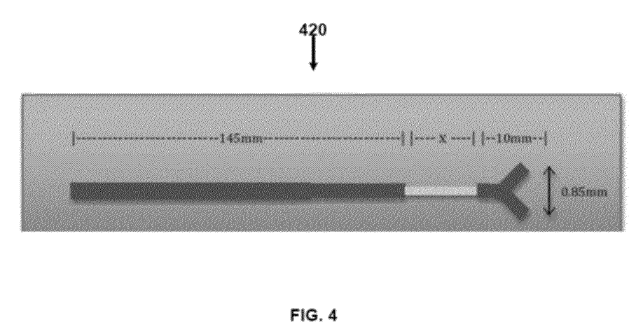

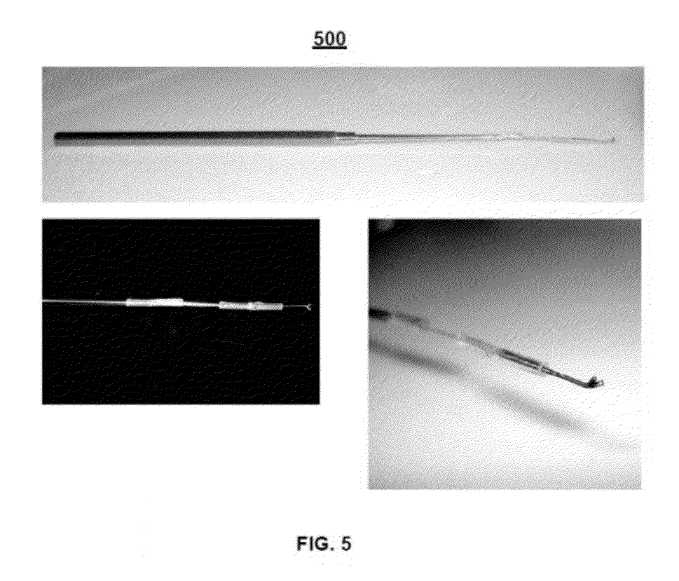

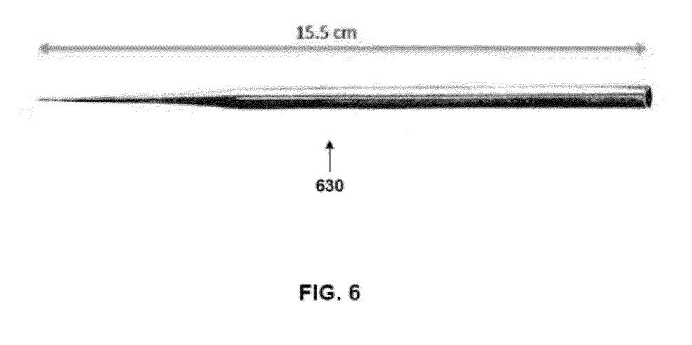

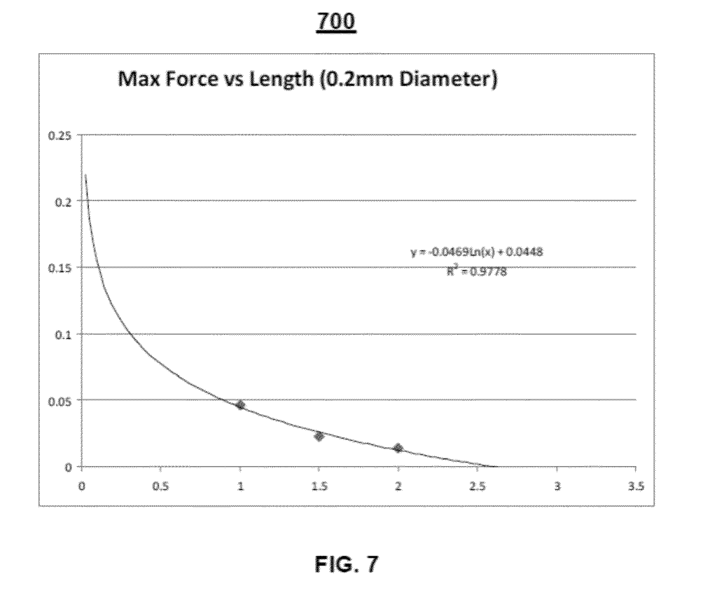

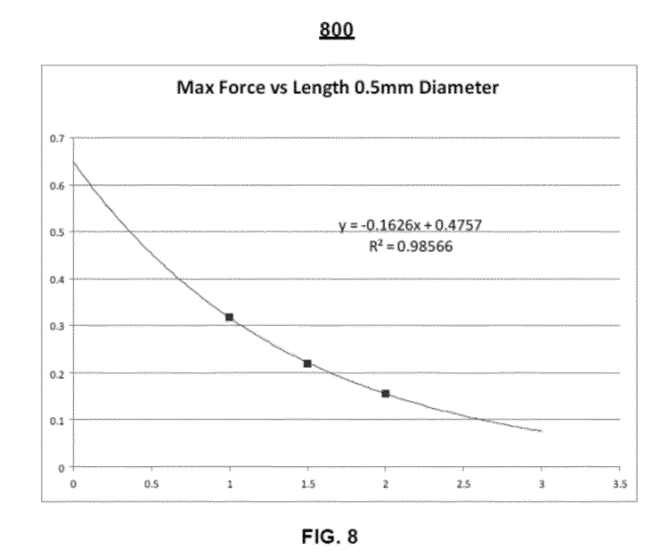

FIG. 4 shows a partial view of a tip portion 420, according to one embodiment of the present disclosure. FIG. 5 shows partial views of a device 500, according to one embodiment of the present disclosure. FIG. 6 shows a partial view of a handle portion 630, according to one embodiment of the present disclosure. FIG. 7 shows a plot 700 of a correlation of first quantified values relating to a resilient member according to one embodiment of the present disclosure. FIG. 8 shows a plot 800 of a correlation of second quantified values relating to a resilient member according to one embodiment of the present disclosure.

It will be understood that the instrument or device may not include all of the portions described herein and may also configured to include additional portions. For example, the instrument or device may not include the handle portion 130 and/or base portion 110. The tip portion 120 may be configured to attach to a different surgical instrument, such as catheter.

### Method

In certain embodiments, a method of implanting an article during surgery is provided, including attaching the article to the device described herein and implanting the article, wherein the device does not allow further implantation if a force threshold is reached. In one embodiment, the article is an electrode array and the surgery is a cochlear implant. In another embodiment, a method is provided including inserting a device as described herein to an implanted article and exerting force sufficient to reach a force threshold, wherein if such a force cannot be reached then the article is not properly implanted. In one embodiment, the article can be a prosthesis, such as a bone or cartilage prosthesis. In another embodiment, a method is provided to determine if a system is properly designed including exerting force on the system with a device as described herein, wherein the force exerted is sufficient to reach a force threshold, and if such a force threshold cannot be reached, the system is not properly designed. Such a system can be, for example, a belt in a motor vehicle such as a drive belt and the system can assess tension in such a belt. In other embodiments, the system includes a spring-loaded closure and the method is useful to determine if the spring is exerting sufficient tension on the closure.

While there have been shown several and alternate embodiments of the present disclosure, it is to be understood that certain changes can be made as would be known to one skilled in the art without departing from the underlying scope of the disclosure as is discussed and set forth above and below including claims. Furthermore, the embodiments described above and claims set forth below are only intended to illustrate the principles of the present disclosure and are not intended to limit the scope of the disclosure to the disclosed elements.

What is claimed is:

1. A device for engaging a target of interest, comprising:
a tip portion, wherein the tip portion includes a first end portion a having an engaging member configured to engage a target of interest, an opposite, second end portion, and a body portion defined therebetween, the body portion including a resilient member configured to be elastically compressible up to a predetermined maximum compression level.

2. The device of claim 1,
wherein the predetermined maximum compression level corresponds to a predetermined buckling limit of an elastic rod formed from a predetermined material and having a predetermined length and width, and
wherein the resilient member is formed at least partially of the predetermined material and have a length and width that are substantially similar to the predetermined length and width of the elastic rod.

3. The device of claim 1, further comprising a base portion having a first end portion, an opposite second end portion, and a body portion defined therebetween, wherein the tip portion extends away the first end portion of the base portion and is substantially coaxial with the base portion

4. The device of claim 1, further comprising:
a base portion having a first end portion, an opposite second end portion, and a body portion defined therebetween; and
a handle portion extending away from the second end portion of the base portion and substantially coaxial with the base portion, wherein the handle portion comprises a first end portion, a second end portion, and a body portion defined therebetween.

5. The device of claim 4, wherein the force applied to the at least one of the tip portion, base portion, and handle portion is the net force in a direction substantially parallel to a longitudinal axis of the base portion that is produced from a force applied to the handle portion in a first direction and a force applied to the tip portion in a second direction that substantially opposite the first direction.

5. The device of claim 1, wherein the resilient member is configured such that at least one of the length and width is adjustable to change the predetermined maximum compression level.

6. The device of claim 1, wherein the tip portion is configured such that the resilient member is selectively detachable from the tip portion and such that a different resilient member having a different predetermined maximum compression level is attachable in place of a detached resilient member.

7. The device of claim 1,
wherein the base portion has a longitudinal axis, the base portion including a first receptacle disposed therein along a longitudinal axis, the first receptacle having sidewalls defining a first inner cavity and a first opening at the end of the first end portion of the base portion, and
wherein the first receptacle is configured to receive at least part of the tip portion in the first inner cavity through the first opening.

8. The device of claim 7,
wherein the base portion includes a second receptacle disposed therein along the longitudinal axis, the second receptacle having sidewalls defining a second inner cavity and a second opening at the end of the second end portion of the base portion, and
wherein the second receptacle is configured to receive at least part of a handle portion in the second inner cavity through the second opening.

9. The device of claim 1, wherein the target of interest includes at least a portion of a surgical prosthesis to be placed in a target area of a living subject.

10. The device of claim 1, wherein the target of interest includes at least a portion of a surgical prosthesis that has been placed in a target area of a living subject.

11. A surgical instrument, comprising:
a base portion having a first end portion, an opposite second end portion, and a body portion defined therebetween, the base portion having a longitudinal axis; and
a tip portion extending away from the first end portion of the base portion in a first direction at a predetermined angle relative to the longitudinal axis of the base portion,
wherein the tip portion includes a first end portion having an engaging member configured to engage a target of interest, an opposite, second end portion, and a body portion defined therebetween, the tip portion including a resilient member configured to be elastically compressible up to a predetermined maximum compression level.

12. The surgical instrument of claim 11,
wherein the predetermined maximum compression level corresponds to a predetermined buckling limit of an elastic rod formed from a predetermined material and having a predetermined length and width, and
wherein the resilient member is formed at least partially of the predetermined material and has a length and width that are substantially similar to the predetermined length and width of the elastic rod.

13. The surgical instrument of claim 11,
wherein the force applied to the at least one of the tip portion, base portion, and a handle portion is the net force in a direction substantially parallel to the longitudinal axis of at least one of the base portion, tip portion, and handle portion that is produced from a force applied to the handle portion in a first direction and a force applied to the tip portion in a second direction that is substantially opposite the first direction.

14. The surgical instrument of claim 11, wherein the resilient member is configured such that at least one of the length and width is adjustable to change the predetermined maximum compression level.

15. The surgical instrument of claim 11, wherein the tip portion is configured such that the resilient member is selectively detachable from the tip portion and such that a different resilient member having a different predetermined maximum compression level is attachable in place of a detached resilient member.

16. The surgical instrument of claim 11, wherein the predetermined angle is in a range from about 0 to about 90 degrees.

17. The surgical instrument of claim 11, wherein the predetermined angle is in a range from about 90 degrees to about 180 degrees.

18. The surgical instrument of claim 11,
wherein the base portion includes a first receptacle disposed therein along the longitudinal axis, the first receptacle having sidewalls defining a first inner cavity and a first opening at the end of the first portion of the base portion, and
wherein the first receptacle is configured to receive at least part of the tip portion inside the first inner cavity through the first opening.

19. The surgical instrument of claim 18,
wherein the base portion includes a second receptacle disposed therein along the longitudinal axis, the second receptacle having sidewalls defining a second inner cavity and a second opening at the end of the second end portion of the base portion, and
wherein the second receptacle is configured to receive at least part of a handle portion inside the second inner cavity through the second opening.

20. The surgical instrument of claim 11, wherein the target of interest includes at least a portion of a surgical prosthesis to be placed in a target area of a living subject.
